(19) United States
(12) Patent Application Publication
(10) Pub. No.: US 2011/0172517 A1
(43) Pub. Date: Jul. 14, 2011

(54) METHOD FOR DETERMINING INFORMATION USED AS THE BASIS FOR CALCULATING A RADIOTHERAPY TREATMENT PLAN AND COMBINED MAGNETIC RESONANCE IMAGING/PET DEVICE

(75) Inventor: Sebastian Schmidt, Weisendorf (DE)

(73) Assignee: Siemens Aktiengesellschaft, Munich (DE)

(21) Appl. No.: 13/004,119

(22) Filed: Jan. 11, 2011

(30) Foreign Application Priority Data
Jan. 12, 2010 (DE) 10 2010 004 384.2

(51) Int. Cl. A61B 5/055

(52) U.S. Cl. 600/411

(57) ABSTRACT

A method for determining information used as the basis for calculating a radiotherapy treatment plan. In at least one embodiment, the method includes essentially simultaneous capture of PET data and magnetic resonance imaging data using a combined magnetic resonance imaging/PET device and determination of at least one magnetic resonance imaging data set and at least one PET data set from the data; and determination of a distortion-corrected magnetic resonance imaging data set and an attenuation data set from the magnetic resonance imaging data, whereby PET data is taken into consideration during the distortion correction and/or during the determination of the attenuation data set.

### PRIORITY STATEMENT

The present application hereby claims priority under 35 U.S.C. §119 on German patent application number DE 10 2010 004 384.2 filed Jan. 12, 2010, the entire contents of which are hereby incorporated herein by reference.

### FIELD

At least one embodiment of the invention generally relates to a method for determining information used as the basis for calculating a radiotherapy treatment plan and/or an associated combined magnetic resonance imaging/PET device.

### BACKGROUND

Treatment methods, in particular in the field of tumor therapy, are known in which an irradiation of the target region to be treated with certain doses takes place for example by accelerating particles to certain energy levels in a linear accelerator and bombarding the target region with said particles. In this situation, the target region and the radiation source are frequently moved relative to one another in order to achieve a maximum irradiation effect on the point to be treated, for example the tumor. Such radiotherapy processes are normally described by means of a radiotherapy treatment plan which in particular seeks to take into account the attenuation of the radiation in the human body.

This is of particular relevance with regard to the specific adjustment of the dose for parts of the target region, so-called “dose painting”. For example, in the case of a prostate cancer the entire prostate is no longer irradiated with approx. 70 Gy as is traditional, but a higher dose, for example 78 Gy, is employed in the region of the tumor. Moreover, the dose can be reduced in non-malignant areas.

The varying radiation sensitivity within the tumor in some variants is also incorporated specifically into the planning. Thus, hypoxic tissue has a lower radiation sensitivity and consequently requires a higher dose whereas tissue well supplied with oxygen has a higher radiation sensitivity.

The use of CT images as a basis when producing the radiotherapy treatment plan is known. CT images have the disadvantage however that they offer only minimal soft tissue contrast and practically no information about the function of the tissue.

In this situation, both PET (positron emission tomography) and also magnetic resonance (MR) imaging are more advantageous. Thus, depending on the tracer used, positron emission tomography can provide information relating to metabolism, to hypoxia and to cell proliferation, but only ever one parameter per examination because the different tracers cannot be employed and differentiated simultaneously. In addition to anatomical information, magnetic resonance imaging can also provide information relating to cell density (diffusion-weighted imaging), blood flow (DCE), chemical composition (magnetic resonance spectroscopy) and oxygen supply (BOLD imaging). In addition, a plurality of contrast levels can be determined in a single examination.

A combined use of PET and MR images for radiotherapy treatment planning has however not been meaningfully possible hitherto because a series of restrictions exists.

On the one hand, magnetic resonance imaging is not positionally accurate. This is because the positional assignment in magnetic resonance imaging depends on various factors which cannot be controlled sufficiently precisely. For example, nonlinearities in the gradient system, inhomogeneities in the constant magnetic field or influences of the patient's body itself can lead to distortion of the images, with the result that in the worst case the incorrect target region is irradiated.

Furthermore, magnetic resonance imaging does not deliver any attenuation information. Information relating to the attenuation of the beams in the tissue is however required for radiotherapy treatment planning, in other words the absorption coefficients for the beams employed for the irradiation must be known for each voxel.

A further problem is the limited field of view of magnetic resonance imaging which means that peripherally situated parts of the body, shoulders and arms for example, do not appear in the image. These are however important for the radiotherapy treatment planning because they likewise attenuate the penetrating radiation or these parts are likewise sensitive to radiation and must be taken into consideration during the planning.

Positron emission tomography on the other hand provides only very little anatomical information, or none at all, which means that the PET images cannot be used for planning.

A proposal has been made to use a combined PET/CT device in order to obtain CT images and PET images registered directly with one another. As mentioned above, however, PET images can only ever deliver one parameter per examination.

The amalgamation of images from different modalities by registering said images on top of each other is known in principle. This does however have a high associated risk of errors. Since the patient is relocated between a PET/CT measurement and an MR measurement, organs become displaced which means that the registration can no longer be carried out without any problems. Although special positioning aids, tables or fixing devices for example, are known with which attempts are made to ensure an identical positioning, even these measures are not successful in every case.

In a combined magnetic resonance imaging/PET device, such as is known for example from DE 10 2005 015 071, although PET images and MR images can be captured simultaneously, these then still need to be amalgamated with a CT data set however in order to solve the aforementioned problems in respect of distortion and attenuation correction.

Although methods for distortion correction of MR images are moreover known, for example from DE 195 40 837, in principle these do not however have the capability to specifically compensate completely for the field distortions caused by the patient.

Finally, methods for creating attenuation maps from MR images are known, for example from DE 10 2004 043 889, which are however merely sufficient to permit a correction of PET images. They do not offer the precision to enable preparation of a radiotherapy treatment plan based thereon.

### SUMMARY

In at least one embodiment of the invention, a method is specified which enables a sufficiently precise determination of information used as the basis for a radiotherapy treatment plan solely on the basis of PET data and magnetic resonance imaging data captured by way of a combined magnetic resonance imaging/PET device.

In at least one embodiment of the invention, the method includes:

essentially simultaneous capture of PET data and magnetic resonance imaging data using a combined magnetic resonance imaging/PET device and determination of at least one magnetic resonance imaging data set and at least one PET data set from the data,

determination of a distortion-corrected magnetic resonance imaging data set and an attenuation data set from the magnetic resonance imaging data, whereby PET data is taken into consideration during the distortion correction and/or during the determination of the attenuation data set.

The method according to at least one embodiment of the invention thus first proposes that PET data and magnetic resonance imaging data be simultaneously captured for the patient ultimately receiving radiotherapy. This PET data and magnetic resonance imaging data ideally encompasses the entire body of the patient, the entire field of view for magnetic resonance imaging data in the case of too small a field of view. For the first time it is now proposed to use both magnetic resonance imaging data and also PET data for calculating the distortion correction and/or the attenuation data set. The data sources which are in any case registered with one another advantageously mutually complement one another to the extent that a more precise dose distribution is possible whilst taking into consideration the biological characteristics of the tissue, and planning errors can be avoided, in particular those which occur as a result of moving the patient for different modalities. As a result of their differing recording techniques, magnetic resonance imaging data and PET data in particular namely contain a wide variety of important and useful information concerning the production of a radiotherapy treatment plan, in particular in respect of the “dose painting” method.

A radiotherapy treatment plan can consequently be determined from the PET data set, the distortion-corrected magnetic resonance imaging data set and from the attenuation data set. In this situation, provision can be made for example such that the distortion-corrected magnetic resonance imaging data set and the PET data set are displayed superimposed, for example in a false color representation. It is then firstly possible to mark the target region, which for example may contain a tumor. Tumor areas are recognizable for example as a result of a high metabolic activity in the FDG PET or a high contrast agent absorption of a magnetic resonance imaging contrast agent.

It is thus possible to segment the target region. Regions which exhibit a particularly high or a particularly low radiation sensitivity are subsequently segmented and assigned accordingly. The special advantage of using simultaneously captured magnetic resonance imaging data and PET data is reflected here. Hypoxic regions can thus for example be rendered visible by way of BOLD imaging in magnetic resonance imaging or by administering the F-MISO tracer in PET. Said hypoxic regions exhibit a reduced radiation sensitivity. A high cell division activity can be made visible from diffusion magnetic resonance images or when using the FLT tracer in PET. This points to an increased radiation sensitivity.

The same applies to regions having a high blood flow, which can be made visible by perfusion magnetic resonance imaging, and regions having a high choline content or a high choline/citrate ratio, which can be identified by magnetic resonance spectroscopy. In general it can therefore be stated that regions of varying radiation sensitivity can be identified, in particular segmented, from the PET data set and/or the distortion-corrected magnetic resonance imaging data set. A desired radiation value, in particular a dose, can then be assigned to each of these regions as a basis for the radiotherapy treatment plan.

The information thus determined together with the attenuation data set serve as input values for calculating the radiotherapy treatment plan. Methods with which a radiotherapy treatment plan can be calculated are widely known in the prior art and do not need to be described here in detail. For example, Monte Carlo simulations and an iterative optimization of the radiotherapy treatment plan can be used.

Evidently the use of the improved distortion-corrected magnetic resonance imaging data offers an excellent starting point for being able to detect regions of special radiation sensitivity. Together with the attenuation data set, it is then possible to produce therefrom a highly precise radiotherapy treatment plan taking into consideration the biological characteristics of the tissue.

As already mentioned, a wide variety of tissue characteristics can be determined by PET and magnetic resonance imaging. For this purpose, certain tracers or recording techniques are to be used in each case. Provision can be made for example such that the PET data is captured after administration of a PET tracer, in particular FDG or F-MISO or FLT or F-uracil or 11C-choline or 11C-methionine, and/or the magnetic resonance imaging data is captured using at least one recording technique, in particular T1-weighted and/or T2-weighted and/or diffusion-weighted and/or using a BOLD technique and/or using a spectroscopy technique and/or using recording techniques having a low echo time.

In an example embodiment, provision can be made for example such that the PET data is captured using FDG as the tracer and MR data is captured in order to form three data sets T1-weighted, T2-weighted and diffusion-weighted (DWI—diffusion weighted imaging). With regard to diffusion-weighted magnetic resonance imaging, it is ultimately the movement along the gradient which is analyzed. Very dense tissue is present in a tumor, which means that the mean free path is rather small, whereas for example in a blister, in which principally water is present, a large diffusion path is possible. Naturally, depending on the information which is most useful for the specific radiotherapy treatment planning, other combinations of imaging techniques and tracers are conceivable. Measuring sequences having very short echo times are known for example under the names UTE, RASP or SWIFT.

In a further embodiment of the present invention, provision can be made such that for the purpose of distortion correction the magnetic resonance imaging data set is elastically registered onto the PET data set, in particular on the basis of landmarks defined in the PET data set and the magnetic resonance imaging data set. For example, a PET data set captured using the tracer FDG contains a wide variety of anatomical information relating to the location of different organs in which landmarks can be defined which can be superimposed with corresponding landmarks in the magnetic resonance imaging data set by “deforming” the magnetic resonance image. In this situation, the regions between the landmarks can be adapted through interpolation. Particularly advantageously, provision can be made such that the registration, in particular a deformation of the magnetic resonance imaging data set occurring within the framework of the registration, occurs taking account of additional parameters describing the positional accuracy of the magnetic resonance imaging data. Thus it is known as background information for example that the positional accuracy tends to be high close to the isocenter of the magnetic resonance imaging system. Provision can then be made such that only slight deformations are permitted there.

The reverse case is demonstrated at the periphery where large deformations can then be entirely possible. In this manner, the ultimately known information concerning the local positional accuracy of the magnetic resonance imaging system is utilized. The result of the registration described here is then a positionally accurate, in other words distortion-corrected, magnetic resonance imaging data set which can be used for the planning.

Preferably, in order to determine the attenuation data set an initial attenuation map can in the first instance be determined through segmentation of the magnetic resonance imaging data set or registration of the magnetic resonance imaging data set onto an atlas, whereby attenuation values and/or density values are assigned to the respective segments, whereupon the attenuation data set is determined through adaptation and/or extension of the initial attenuation map. In particular, the already distortion-corrected magnetic resonance imaging data set is naturally used for this purpose. Such a first attenuation map (μ map) can for example be produced by segmenting the magnetic resonance imaging data set and assigning density values to the individual segments. It is also conceivable for an anatomical atlas to be registered onto the magnetic resonance imaging data set. Suchlike is described for example in DE 10 2004 043 889 A1, the entire contents of which are hereby incorporated herein by reference.

After it has been determined, this first attenuation map is adapted on the basis of the PET data. In this situation, provision can advantageously be made such that the attenuation map is supplemented from the PET data by anatomical information, in particular a surface contour, in those regions not captured, or only captured in poor quality, by the magnetic resonance imaging data set. In this manner it is possible for example to add the surface contour of the body of a patient from the PET data. Since PET has a greater field of view than the magnetic resonance imaging system, the body surface of the scanned patient is represented in its entirety in the PET data set whereas peripheral portions are missing or are represented only in poor quality in the magnetic resonance imaging data set. A supplementation can consequently take place, using the PET data. Fixed attenuation values for example can then be assumed for the added regions.

Furthermore, provision can be made such that the initial attenuation map is adapted by way of an iterative method, using the PET data. In this situation, for example, the first attenuation map can be utilized as the initialization basis for a so-called MLEM algorithm (maximum likelihood expectation maximization). In this situation, a model can be prepared for example which is adapted such that it best matches the measured PET data.

In this situation, a further possibility for adapting the attenuation map is for example the ESF method (“Emission Segmentation by Fuzzy Inference”) described in the dissertation by Kilian Bilger, “Verkürzung der Transmissionszeit bei einem Positronen-Emissions-Tomographen (PET) durch die segmentierte Schwächungskorrektur” (Reduction of transmission scan time on a Positron Emission Tomograph (PET) by segmented attenuation correction), Chapter 2.3, the entire contents of which are hereby incorporated herein by reference. In contrast to what is described there, with regard to the method according to the invention the mask values are determined not from the PET data but from the magnetic resonance imaging data set. In this case, the determination of the attenuation data set is thus carried out using an ESF method, whereby mask values are determined from the magnetic resonance imaging data or the initial attenuation map is used as a mask.

Provision can advantageously be made such that the attenuation data set relating to PET photons is converted into the energy used for the radiotherapy. If the attenuation data set occurring in the result relates to the attenuation of PET photons (energy approximately 500 keV), then a conversion of the attenuation values contained therein to high-energy radiation, as is produced for example by a linear accelerator, can consequently still take place.

In addition to the method, in at least one embodiment of the invention also relates to a combined magnetic resonance imaging/PET device, designed to simultaneously capture magnetic resonance imaging data and PET data, which includes a control unit designed to carry out the method according to at least one embodiment of the invention. All the statements made with respect to the method according to at least one embodiment of the invention can be applied in analogous fashion to the device according to at least one embodiment of the invention.

Combined magnetic resonance imaging/PET devices which permit the simultaneous capture of magnetic resonance imaging data and PET data are widely known and do not need to be described in further detail here. They include a PET capture device which frequently comprises a PET detector ring which can be pushed or slid into a patient examination area of a magnetic resonance imaging system. Other geometries are however also conceivable.

### BRIEF DESCRIPTION OF THE DRAWINGS

Further advantages and details of the present invention will emerge from the exemplary embodiments described in the following and with reference to the drawings. In the drawings:

FIG. 1 shows a flowchart of the method according to an embodiment of the invention, and

FIG. 2 shows a combined magnetic resonance imaging/PET device.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

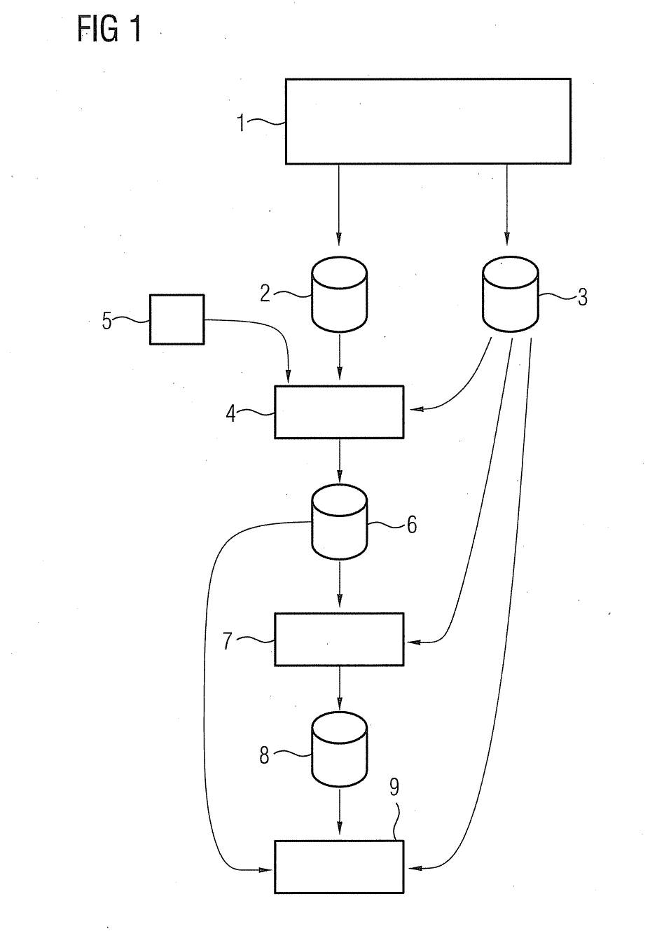

FIG. 1 shows a flowchart of the method according to the invention, which is carried out with the aid of a combined magnetic resonance imaging/PET device. Firstly, a simultaneous capture of magnetic resonance imaging data and PET data takes place in a step 1. In this situation, FDG for example can be used as the tracer, and a T1-weighted technique, a T2-weighted technique and a diffusion-weighted technique as magnetic resonance imaging techniques. At least one MR data set 2 and at least one PET data set 3 are then determined from the data thus captured.

In a step 4 the magnetic resonance imaging data set 2 is distortion-corrected, whereby the PET data set 3 is taken into consideration. To this end, a registration of the magnetic resonance imaging data set 2 to the PET data set 3 on the basis of anatomical landmarks is first performed. This is an elastic registration process, which means that the magnetic resonance imaging data set 2 is adapted through deformation such that the landmarks can be superimposed. In this situation, the regions between the landmarks are adapted through interpolation.

Provision is made in this situation such that additional information indicated at 5 which describes the expected positional accuracy of the magnetic resonance imaging data is taken into consideration. The deformation can be limited by way of this data. The magnetic resonance imaging data close to the isocenter thus has very high positional accuracy for the most part, which means that only very minor deformations are permitted. At the periphery of the field of view the positional accuracy is however very low, which means that major deformations can also be permitted.

The result of the registration thus performed is a distortion-corrected magnetic resonance imaging data set 6. It should be noted that if a plurality of magnetic resonance image recording techniques is used all the magnetic resonance imaging data sets originating therefrom are naturally also correspondingly distortion-corrected, with the result that then only distortion-corrected magnetic resonance imaging data sets are still present. These distortion-corrected magnetic resonance imaging data sets also already represent a first basis for the subsequently occurring preparation of a radiotherapy treatment plan.

Firstly, however, in a step 7 in the method according to the invention an attenuation data set 8 is additionally determined. To this end, provision is firstly made for determining an initial attenuation map from the distortion-corrected magnetic resonance imaging data set 6 by segmenting the magnetic resonance imaging data set—as far as possible—according to different tissue types, to which corresponding linear attenuation coefficients can then be assigned. Thus there are then several possible ways of determining an attenuation data set 8 herefrom using the PET data from the PET data set 3.

Provision can be made on the one hand such that the PET data is used in order to add regions which are not contained in the field of view of the magnetic resonance imaging system. These can comprise for example arms and shoulders which on account of the larger field of view of the PET scanning device are still clearly recognizable there. The added regions can then likewise be provided with linear attenuation coefficients, with the result that the attenuation data set 8 is produced.

By preference, it is however possible to utilize a method using a MLEM algorithm. Several options are available to this end. On the one hand, such a MLEM algorithm can be initialized through the initial attenuation map in order to then obtain the attenuation data set 8 herefrom. It is however also conceivable to start from a model describing an average attenuation value data set, permitted deviations therefrom and the location and orientation or size of the attenuation data set, whereby a model instance is produced which is matched to the PET data, for which purpose a MLEM algorithm can again be used. This MLEM algorithm is further restricted by the initial attenuation map.

Finally, it is also conceivable to use an ESF method (emission segmentation by fuzzy inference) known from the prior art, whereby mask values are derived from the distortion-corrected magnetic resonance imaging data set 6.

In all these cases a very precise attenuation data set 8 is determined, which however in the event of use of a MLEM algorithm, possibly however also in the event of use of other algorithms, relates to the energy of the PET photons or to a different energy. It may then be necessary to convert the attenuation data set 8 to the energy subsequently utilized for the radiotherapy.

With the PET data set 3, the distortion-corrected magnetic resonance imaging data set 6 and the attenuation data set 8 important information is now available which is ultimately used for calculating a radiotherapy treatment plan in step 9. To this end, the at least one distortion-corrected magnetic resonance imaging data set 6 and the PET data set 3 are represented superimposed in a false color representation. On account of the special recording techniques and the tracer used, in addition to locating the tumor to be irradiated in the target region it is possible to segment the most diverse tissue regions. Thus, in images captured using the PET tracer F-MISO or magnetic resonance imaging data sets produced by the BOLD technique, hypoxic tissues are to be recognized which exhibit a reduced radiation sensitivity. Analogously, high levels of cell division activity can be ascertained from diffusion magnetic resonance imaging data sets or PET images captured using the FLT tracer, which speaks for a higher radiation sensitivity and suchlike.

Correspondingly segmented regions are therefore marked with regard to their radiation sensitivity. This information is used jointly with the attenuation data set 8 as input values for calculating the radiotherapy treatment plan, which can take place for example by way of Monte Carlo simulations and iterative optimizations.

This will be briefly explained in detail by way of example of a prostate radiotherapy. Here the following images were captured using a combined magnetic resonance imaging/PET device in a single examination: After administering the tracer F-MISO a PET data set was recorded, additionally a T2-weighted magnetic resonance imaging data set and a T1-weighted magnetic resonance imaging data set after administering a contrast agent. In this situation, for example, a lesion which absorbs contrast agent, situated on the left-hand side within the prostate, can be revealed in the T2-weighted magnetic resonance imaging data set: A tumor. Within this lesion, a hypoxic area is revealed in the PET data set. The prostate is therefore segmented in this example and a dose of 70 Gy for example is assigned. A higher dose, 75 Gy for example, is assigned to the tumor area. An even higher dose, 80 Gy for example, is assigned to the hypoxic part of the tumor. Optimum effectiveness is thus achieved whilst minimizing the effects on surrounding tissues (rectum, nerves, for example).

It should be pointed out once again that—depending on the information desired—other combinations of PET tracer and magnetic resonance image recording techniques can naturally also be used.

By using the method according to an embodiment of the invention, the information required for preparation of a highly precise radiotherapy treatment plan can in general consequently be successfully obtained solely from magnetic resonance imaging data and PET data by taking into consideration both magnetic resonance imaging data and also PET data during the distortion correction of the magnetic resonance imaging data and also during the determination of an attenuation data set.

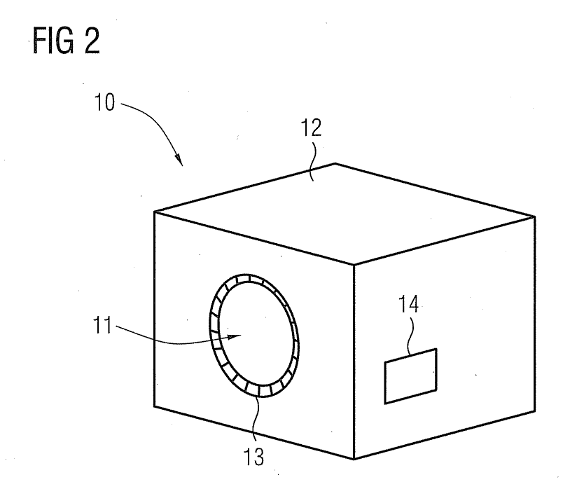

FIG. 2 shows a magnetic resonance imaging/PET device 10 according to an embodiment of the invention. With this it is possible to simultaneously capture magnetic resonance imaging data and PET data by inserting a PET detector ring 13 into the patient recording 11 of a magnetic resonance imaging system 12.

The data capture operation is controlled by a control unit 14. This is designed for carrying out the method according to an embodiment of the invention, such that all the data which is required for preparing a radiotherapy treatment plan can be determined directly on the combined magnetic resonance imaging/PET device 10.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, non-transitory computer readable medium and non-transitory computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory storage medium or non-transitory computer readable medium, is adapted to store information and is adapted to interact with a data processing facility or computer device to execute the program of any of the above mentioned embodiments and/or to perform the method of any of the above mentioned embodiments.

The non-transitory computer readable medium or non-transitory storage medium may be a built-in medium installed inside a computer device main body or a removable non-transitory medium arranged so that it can be separated from the computer device main body. Examples of the built-in non-transitory medium include, but are not limited to, rewriteable non-volatile memories, such as ROMs and flash memories, and hard disks. Examples of the removable non-transitory medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### LIST OF REFERENCE CHARACTERS

1 Step
2 Magnetic resonance imaging data set
3 PET data set
4 Step
5 Additional information
6 Magnetic resonance imaging data set
7 Step
8 Attenuation data set
9 Step
10 Magnetic resonance imaging/PET device
11 Patient recording
12 Magnetic resonance imaging system
13 PET detector ring
14 Control unit

What is claimed is:

1. A method for determining information used as the basis for calculating a radiotherapy treatment plan, comprising:
essentially simultaneously capturing PET data and magnetic resonance imaging data using a combined magnetic resonance imaging/PET device;
determining at least one magnetic resonance imaging data set and at least one PET data set from the captured data; and
determining a distortion-corrected magnetic resonance imaging data set and an attenuation data set from the magnetic resonance imaging data, whereby the PET data is taken into consideration during at least one of the distortion correction determination and the determination of the attenuation data set.

2. The method as claimed in claim 1, wherein a radiotherapy treatment plan is determined from the PET data set, the distortion-corrected magnetic resonance imaging data set and from the attenuation data set.

3. The method as claimed in claim 1, wherein at least one of
the PET data is captured after administration of a PET tracer, and
the magnetic resonance imaging data is captured using at least one of at least one recording technique, a BOLD technique, a spectroscopy technique, and recording techniques having a low echo time.

4. The method as claimed in claim 1, wherein, for the distortion correction, the magnetic resonance imaging data set is elastically registered onto the PET data set.

5. The method as claimed in claim 4, wherein the registration occurs taking account of additional parameters describing the positional accuracy of the magnetic resonance imaging data.

6. The method as claimed in claim 1, wherein, in order to determine the attenuation data set, an initial attenuation map is determinable through segmentation of the magnetic resonance imaging data set or registration of the magnetic resonance imaging data set onto an atlas, whereby at least one of attenuation values and density values are assigned to the respective segments, whereupon the attenuation data set is determined through at least one of adaptation and extension of the initial attenuation map.

7. The method as claimed in claim 6, wherein the attenuation map is supplemented from the PET data by anatomical information in those regions not captured, or only captured in poor quality, by the magnetic resonance imaging data set.

8. The method as claimed in claim 6, wherein the initial attenuation map is adapted by way of an iterative method, using the PET data.

9. The method as claimed in claim 6, wherein the adaptation of the initial attenuation map takes place using an ESF method, whereby mask values are determined from the magnetic resonance imaging data.

10. The method as claimed in claim 1, wherein the attenuation data set relating to PET photons is converted into the energy used for the radiotherapy.

11. A combined magnetic resonance imaging/PET device, designed to simultaneously capture magnetic resonance imaging data and PET data, comprising:
a control unit designed to
essentially simultaneously capture PET data and magnetic resonance imaging data using a combined magnetic resonance imaging/PET device,
determine at least one magnetic resonance imaging data set and at least one PET data set from the captured data, and
determine a distortion-corrected magnetic resonance imaging data set and an attenuation data set from the magnetic resonance imaging data, whereby the PET data is taken into consideration during at least one of the distortion correction determination and the determination of the attenuation data set.

12. The method as claimed in claim 3, wherein the PET tracer includes FDG or F-MISO or FLT or F-uracil or 11C-choline or 11C-methionine.

13. The method as claimed in claim 3, wherein the at least one recording technique includes at least one of T1-weighted, T2-weighted and diffusion-weighted.

14. The method as claimed in claim 12, wherein the at least one recording technique includes at least one of T1-weighted, T2-weighted and diffusion-weighted.

15. The method as claimed in claim 4, wherein, for the distortion correction, the magnetic resonance imaging data set is elastically registered onto the PET data set on the basis of landmarks defined in the PET data set and the magnetic resonance imaging data set.

16. The method as claimed in claim 4, wherein a deformation of the magnetic resonance imaging data set, occurring within the framework of the registration, occurs taking account of additional parameters describing the positional accuracy of the magnetic resonance imaging data.

17. The method as claimed in claim 7, wherein the anatomical information includes a surface contour.

18. The method as claimed in claim 7, wherein the initial attenuation map is adapted by way of an iterative method, using the PET data.

19. The method as claimed in claim 7, wherein the adaptation of the initial attenuation map takes place using an ESF method, whereby mask values are determined from the magnetic resonance imaging data.
